(19) United States
(12) Patent Application Publication
(10) Pub. No.: US 2021/0030838 A1
(43) Pub. Date: Feb. 4, 2021

(54) CHEMOTAXIS-POTENTIATING PEPTIDES AND USES THEREOF

(71) Applicant: University of Copenhagen, Copenhagen (DK)

(72) Inventors: Peter Johannes HOLST, Søborg (DK); Line BARINGTON, Copenhagen N (DK); Olav LARSEN, Kgs. Lyngby (DK); Astrid Sissel JØRGENSEN, Copenhagen NV (DK); Gertrud Malene HJORTØ, Copenhagen V (DK); Mette Marie ROSENKILDE, Hellerup (DK)

(21) Appl. No.: 16/638,162

(22) PCT Filed: Sep. 20, 2018
(86) PCT No.: PCT/EP2018/075442

§ 371 Date: Feb. 11, 2020

(30) Foreign Application Priority Data
Sep. 21, 2017 (EP) 17192392.3

(51) Int. Cl. A61K 38/16; A61K 41/00; A61K 47/69; A61K 35/12

(52) U.S. Cl. CPC A61K 38/16; A61K 41/00; A61K 45/06; A61K 35/12; A61K 47/6929

(57) ABSTRACT

The present invention relates to peptides which are capable of potentiating the chemotactic potential of glycosaminoglycan binding chemokines, and to the use of said chemotaxis-potentiating peptides in the treatment of cancer.

### TECHNICAL FIELD

The present invention relates to peptides which are capable of potentiating the chemotactic potential of glycosaminoglycan binding chemokines, and to the use of said chemotaxis-potentiating peptides in the treatment of cancer.

### BACKGROUND

Cancer is a major health problem. Despite temporary effect of many anti-cancer therapies, relapse often occurs especially in patients with advanced stage cancer. Exploitation of the immune system in anti-cancer therapies is increasingly being explored as supplement to conventional cytotoxic cancer therapy or as less-toxic alternatives to conventional therapy. Isolation and ex vivo expansion of tumor infiltrating T-cells (T-cells residing in the patient's tumor, already primed to recognize the tumor as a target), has been explored to maximize immune system attack on the cancer.

CCR7 is a 7 transmembrane (7TM) G protein-coupled receptor (GPCR) expressed on naïve T-cells (immune effector cells) and on mature dendritic cells (antigen presenting cells (DCs)). Two endogenous ligands of CCR7 have been identified, the chemokines CCL19 and CCL21.

CCR7 and the endogenous ligand CCL21 coordinate the meeting between antigen presenting DCs and naïve T-cells. The meeting normally takes place in the lymph nodes, initiating a T-cell mediated immune response against the antigen presented by the DCs. The meeting can also take place in a tumor. The very fact that CCL21 acts to bring together DCs and T-cells to initiate immune responses makes it an attractive molecule for guiding intra-tumoral DC mediated activation of T-cells.

It has previously been suggested that direct intra-tumoral CCL21 injections could increase the number of tumor infiltrating T-cells (TILs) directed against antigens characterizing the otherwise poorly immunogenic tumors.

WO 2002/085286 teaches the application of CCL21 for immunotherapy of cancer. Administering CCL21 to tumor sites resulted in reduced growth of the tumour cells.

WO 2016/073759 teaches the application of CCL21 for cancer combination immunotherapy.

CCL21 is, however, a relatively weak chemo-attractant.

Hjortø et al. Front Immunol. 2016 teaches that deletion of the CCL21 C-terminal tail, to provide tailless CCL21, results in a chemokine with improved chemotactic effect but a lowered CCR7-mediated signalling. It further suggests that the C-terminal tail of full length CCL21 is involved in GAG-binding to the surface of dendritic cells.

Schumann et al. Immunity 2010 teaches that removal of the C-terminal tail from CCL21 results in increased chemotaxis.

Vanheule et al. Front Immunol. 2017 teaches that a CXCL9 C-terminal peptide (CXCL9(74-103)) is capable of binding GAGs. The GAG binding of this peptide was shown to inhibit CXCL8-induced neutrophil migration through inhibition of the GAG-binding of this chemokine.

In order to obtain better cancer immunotherapy, there is a need in the art to improve CCL21 induced chemotaxis, thereby potentiating tumor infiltrating T-cell (TIL) recruitment to tumors resulting in improved anti-cancer efficacy. The peptides of the present invention are shown to provide this improvement in CCL21 induced chemotaxis and are shown to result in reduced tumor growth in a mouse model.

### SUMMARY

The present invention relates to chemotaxis-potentiating peptides which are capable of binding to glycosaminoglycans (GAGs) and potentiate the chemotactic potential of GAG-binding chemokines, and to the use of said chemotaxis-potentiating peptides in the treatment of cancer. The peptides of the present disclosure comprise or consist of the C-terminal tail of human CCL21 and variants, fragments or variants of fragments thereof.

The present inventors have surprisingly found that the C-terminal tail of CCL21 and variants, fragments or variants of fragments thereof are capable of potentiating the chemotactic potential of CCL21 and reduce tumor growth in an in vivo tumor mouse model. The observed improvement of chemotactic potential of endogenous or supplemented CCL21 in the tumor should provide an increase in the number of tumor infiltrating T-cells (TILs) directed against antigens characterizing the tumor.

The peptides of the present disclosure are hypothesized to exert their effect by binding to the glycosaminoglycans (GAGs) and thereby release GAG-bound CCL21, which increases the soluble concentration of CCL21 and the amount able to bind to, and thus recruit T cells.

It is also hypothesized that binding of the peptides of the present invention to the glycosaminoglycans (GAGs) will result in release of intra-tumoral CCL21 which will decrease the autocrine growth advantage induced by the presence of intra-tumoral CCL21 in the tumor.

In one aspect of the present disclosure, peptides are provided wherein the peptide comprises analogues, fragments or analogues of fragments of the C-terminal tail of human CCL21, wherein the peptides are capable of binding to glycosaminoglycans.

In a second aspect of the present disclosure, a method of treatment of cancer, utilizing said peptides, is provided.

In a third aspect of the present disclosure, a method of potentiating immune activation in a tumor, utilizing said peptides, is provided.

In a fourth aspect, a method of potentiating the chemotactic potential of CCL21, utilizing said peptides, is provided.

### DESCRIPTION OF DRAWINGS

FIG. 1

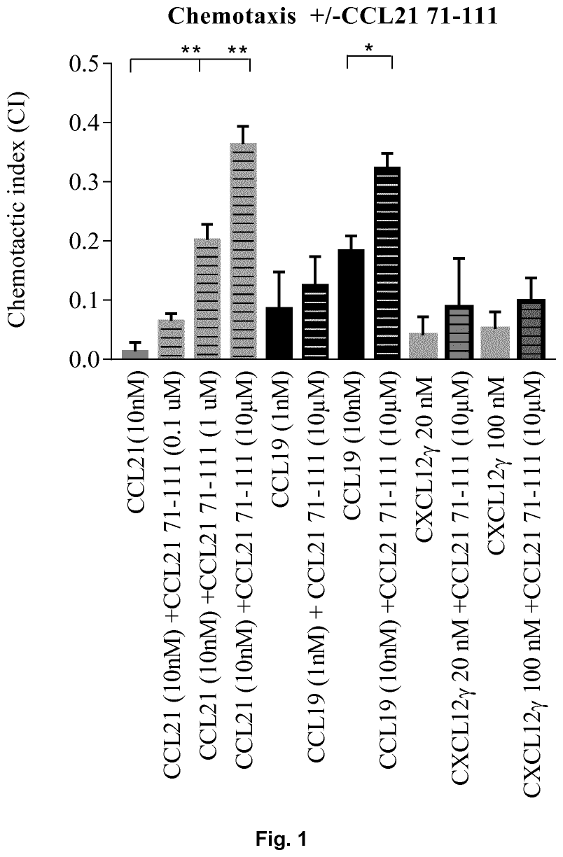

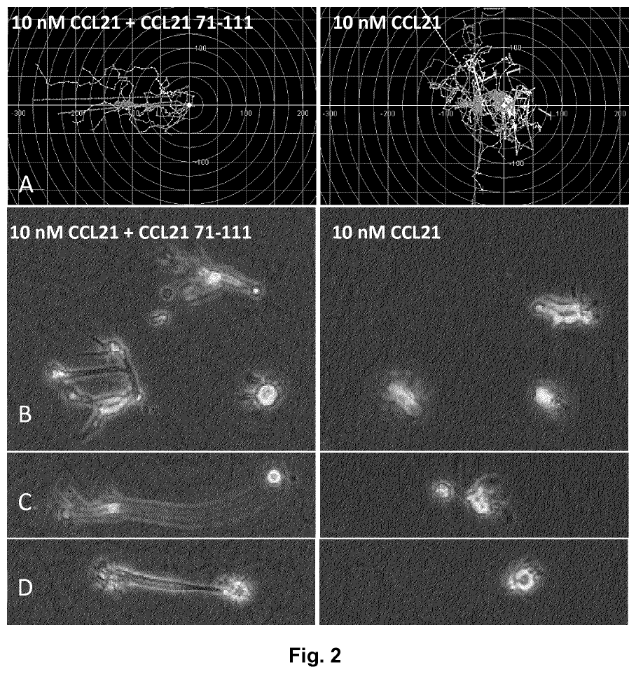

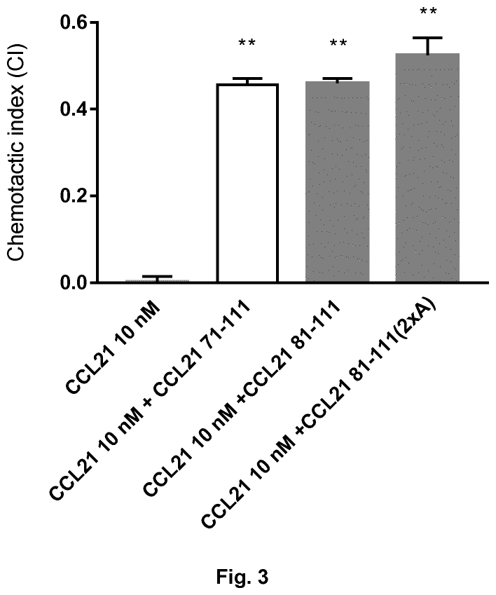

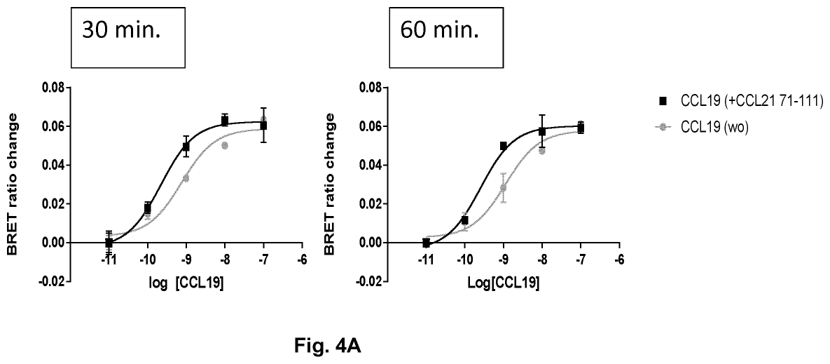

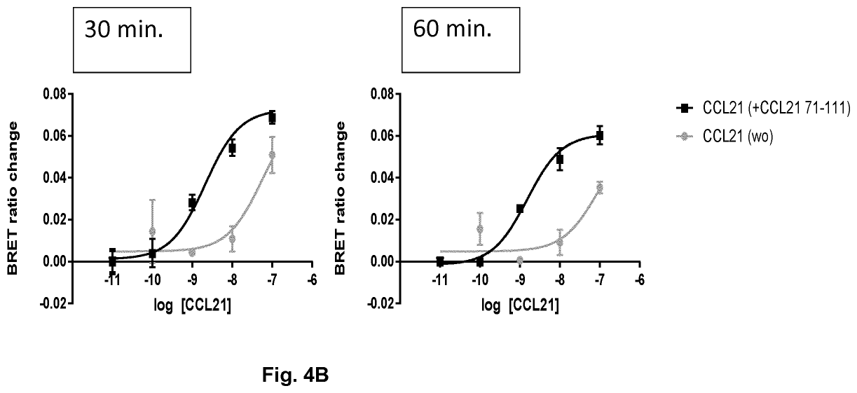

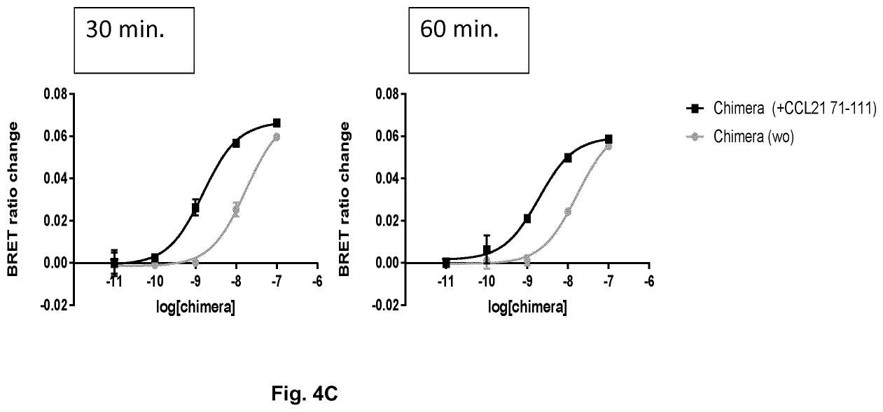

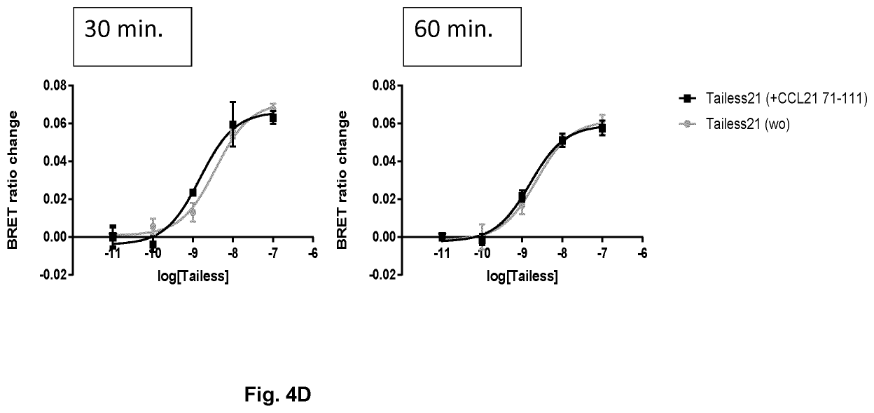

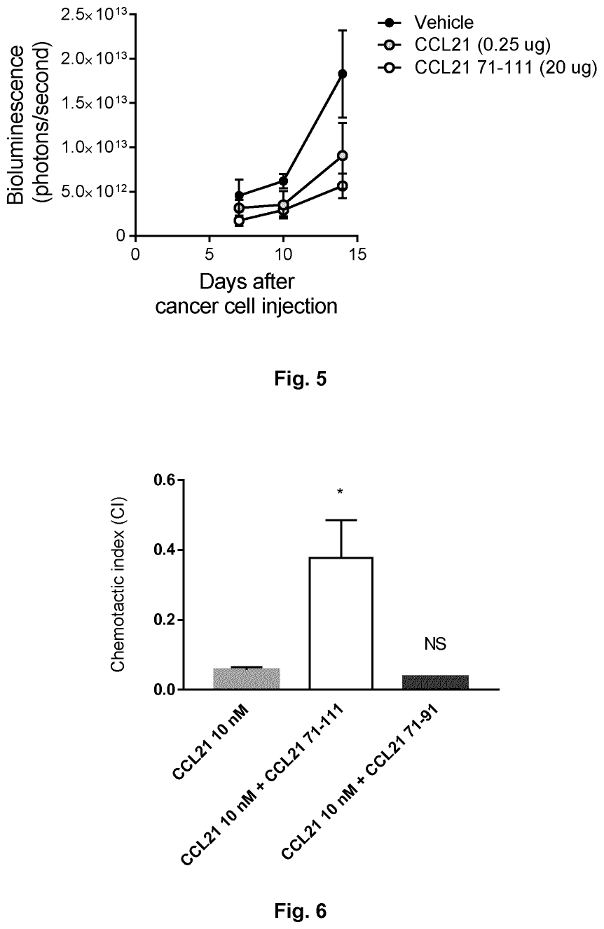

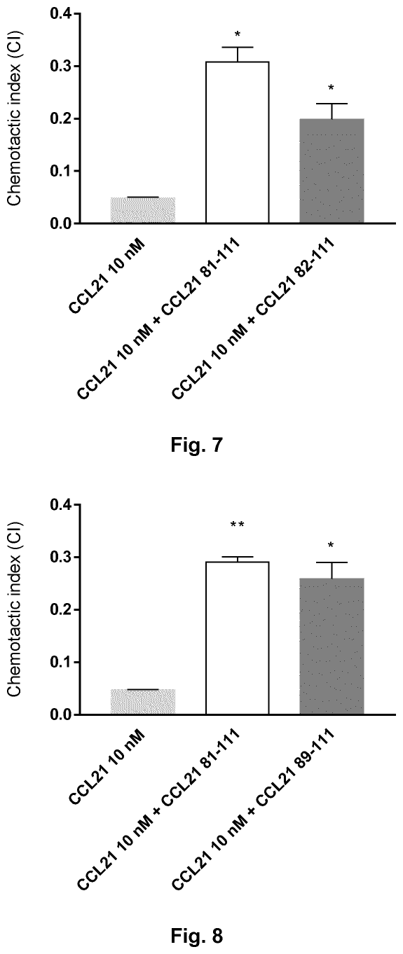

Effect of CCL21 Tail peptide (CCL21 71-111, SEQ ID NO: 1) on chemotaxis of monocyte-derived human DCs (moDC). It was found that CCL21 71-111 potentiates chemotaxis of monocyte derived human DCs (moDC) induced by 10 nM CCL21 in a dose dependent manner, up to 26 fold (FIG. 1, column 1-4). The effect of CCL21 71-111 (10 uM) on chemotaxis induced by CCL19 (1 and 10 nM) as well as chemotaxis induced by CXCL12gamma (20 and 100 nM) was also tested. CCL21 71-111 was found to potentiate the effect of CCL19 by only two fold (column 5-8), and there was no strong chemotaxis signal potentiated by CXCL12gamma induced migration (column 9-12).

FIG. 2

The effect of CCL21 tail peptide (CCL21 71-111, SEQ ID NO: 1) on moDC chemotaxis was reflected by the spidergrams extracted during chemotaxis analysis (FIG. 2A) (chemokine source applied on left side) as well as being readily observable by the change in moDC shape/morphology during migration (FIG. 2B-D).

FIG. 3

Three CCL21 tail peptides were tested on CCL21 induced chemotaxis of monocyte derived human DCs (moDC). The tested peptides were CCL21 Tail peptide (CCL21 71-111, SEQ ID NO: 1), a peptide that is N-terminally truncated by 10 amino acids compared to CCL21 71-111 (CCL21 81-111, SEQ ID NO: 4), and CCL21 81-111 having two amino acid substitutions corresponding to D13A and E33A (CCL21 81-111 (2×A), SEQ ID NO: 5) All three peptides were found to induce chemotaxis with similar potency.

FIG. 4

CCL21 Tail peptide (CCL21 71-111, SEQ ID NO: 1) was tested for its effect on CCL19 WT, CCL21 WT, CCL19 chimera (CCL19 with CCL21 Tail peptide attached at the C-terminus, chimera) and tailless CCL21 (tailless or tailless 21)-induced CCR7 signalling via Gαi. Signalling is detected as a decrease in cAMP which is reflected by an increase in BRET ratio. As reported previously by the inventors and others, CCL19 is more potent than CCL21 in inducing G-protein signalling via CCR7. Tailless CCL21 resembles CCL19, showing essentially no difference in CCR7 signalling in the presence (black curve) or absence (grey curve) of CCL21 71-111 (FIGS. 4A and 4D). In contrast, full-length CCL21 and CCL19chimera (both containing the C-terminal tail of CCL21) showed increased CCR7 signalling in the presence of CCL21 71-111 (black curves), compared to the ligands in the absence of CCL21 71-111 (grey curves) (FIGS. 4B and 4C).

FIG. 5

CCL21 Tail peptide (CCL21 71-111, SEQ ID NO: 1) was tested for its effect on in vivo cancer growth in mice. Female Balb/c mice were injected intraperitoneally with 1.5×10⁵ CT26-FL3-luc cancer cells on day 0. On days 1, 2, and 3, the mice were injected intraperitoneally with either 0.25 μg murine CCL21, 20 μg CCL21 71-111, or vehicle (PBS). The number of mice per group was 5-7, and data is shown as mean±SEM. Both CCL21 and CCL21 71-111 treatment decreased tumor growth. Treatment with CCL21 71-111 was shown to be more effective than treatment with the chemokine CCL21.

FIG. 6

To investigate the C-terminal requirements for a peptide retaining full potentiating effect on CCL21 induced chemotaxis of monocyte derived human DCs (moDC), we tested a peptide that is C-terminally truncated by 20 amino acids compared to CCL21 71-111 (CCL21 71-91, SEQ ID NO: 36). The tested peptide CCL21 71-91 did not potentiate chemotaxis, whereas CCL21 71-111 retained full potency.

FIG. 7

To investigate the N-terminal requirements for a peptide retaining full potentiating effect on CCL21 induced chemotaxis of monocyte derived human DCs (moDC), we tested a peptide that is N-terminally truncated by 11 amino acids compared to CCL21 71-111 (CCL21 82-111, SEQ ID NO: 6). CCL21 82-111 was compared to CCL21 81-111 (SEQ ID NO: 4) as positive control. Whereas CCL21 81-111, as shown in FIG. 3, retained full potentiating effect, CCL21 82-111, displayed reduced capability to potentiate chemotaxis, although it was still functional.

FIG. 8

To further investigate the N-terminal requirements for a peptide retaining full potentiating effect on CCL21 induced chemotaxis of monocyte derived human DCs (moDC), we tested a peptide that is N-terminally truncated by 18 amino acids compared to CCL21 71-111 (CCL21 89-111, SEQ ID NO: 13). CCL21 89-111 was tested using CCL21 81-111 (SEQ ID NO: 4) as positive control. Similar to CCL21 81-111, CCL21 89-111 was shown to retain full potentiating effect. Thus it seems that not only peptide length, but also truncation site influences peptide function.

### DEFINITIONS

The term “negative amino acid residues”, as used herein, refers to amino acid residues which are de-protonated and have a negative charge at physiological pH. Proteinogenic negative amino acid residues are aspartate and glutamate.

The term “neutral amino acid residues”, as used herein, refers to amino acid residues which have no charge at physiological pH. Proteinogenic neutral amino acid residues are glutamine, asparagine, serine, threonine, tyrosine, cysteine, tryptophan, alanine, isoleucine, leucine, methionine, phenylalanine, valine, proline and glycine.

The term “positive amino acid residues”, as used herein, refers to amino acid residues which are protonated and have a positive charge at physiological pH. Proteinogenic positive amino acid residues are lysine, arginine and histidine.

The term “cytokine”, as used herein, refers to small proteins which have an effect on the behaviour of cells surrounding them and which are important for cell signalling. One subclass of cytokines is chemokines. Pro-inflammatory chemokines are induced during an immune response and functions by recruiting cells of the immune system to a site of infection. Homeostatic chemokines are controlling the migration of cells during tissue maintenance or development.

The term “glycosaminoglycans (GAGs)”, as used herein, refers to long unbranched polysaccharides containing a repeating disaccharide unit. The disaccharide units contain an amino sugar (N-acetylglucosamine or N-acetylgalactosamine) along with a uronic sugar (glucuronic acid or iduronic acid) or galactose. Glycosaminoglycans are located primarily on the surface of cells or in the extracellular matrix. Glycosaminoglycans are also known as mucopolysaccharides.

The term “treatment”, as used herein, refers to the medical management of a patient with the intent to cure, ameliorate, stabilize, or prevent a disease, pathological or clinical condition, or disorder, and may include even minimal changes or improvements in one or more measurable markers of the disease or condition being treated. This term includes active treatment, that is, treatment directed specifically toward the improvement of a disease, pathological condition, or disorder. In addition, this term includes preventative treatment, that is, treatment directed to minimizing or partially or completely inhibiting the development of the associated disease, pathological condition, or disorder; and combination treatment, that is, treatment employed to supplement another specific therapy directed toward the improvement of the associated disease, pathological condition, or disorder. “Treatment” or “treating” does not necessarily indicate complete eradication or cure of the disease or condition, or associated symptoms thereof.

As used herein, the term “individual” can be a vertebrate, such as a mammal, a fish, a bird, a reptile, or an amphibian. Thus, the individual of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the individual is a mammal, preferably a human.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, intradermal administration and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “effective amount”, as used herein, refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts.

The term “anti-cancer agent”, as used herein, refers to an agent which is used to treat malignancies, or cancerous growths. The anti-cancer agent may be used alone, or in combination with other treatments such as chemotherapy, immunotherapy, surgery or radiation therapy.

The term “CCR7-positive cancer”, as used herein, refers to a cancer which expresses CCR7. It is not related to a specific type of cancer but to a state of a given cancer-type, wherein the cancer cells are actively expressing CCR7. Whether a given cancer is CCR7-positive can be determined by the skilled person, e.g. by biopsy followed by expression analysis of the isolated tissue/cells of CCR7.

Sequences

| | | Domains, |
|---|---|---|
| SEQ ID NO | Amino acid sequence | substitutions |
| SEQ ID NO: 1 | PSPQKPAQGC RKDRGASKTG KKGKGSKGCK RTERSQTPKG P | CCL21 71-111 |
| | | (D1-D4) |
| SEQ ID NO: 2 | PSPQKPAQGC RKDRGASKTG KKGKGSKGCK R | CCL21 71-101 |
| | | (D1-D3) |
| SEQ ID NO: 3 | RKDRGASKTG KKGKGSKGCK R | CCL21 81-101 |
| | | (D2-D3) |
| SEQ ID NO: 4 | RKDRGASKTG KKGKGSKGCK RTERSQTPKG P | CCL21 81-111 |
| | | (D2-D4) |
| SEQ ID NO: 5 | RKARGASKTG KKGKGSKGCK RTARSQTPKG P | CCL21 81-111 |
| | | (D13A, E33A) |
| SEQ ID NO: 6 | KDRGASKTG KKGKGSKGCK RTERSQTPKG P | CCL21 82-111 |
| SEQ ID NO: 7 | DRGASKTG KKGKGSKGCK RTERSQTPKG P | CCL21 83-111 |
| SEQ ID NO: 8 | RGASKTG KKGKGSKGCK RTERSQTPKG P | CCL21 84-111 |
| SEQ ID NO: 9 | GASKTG KKGKGSKGCK RTERSQTPKG P | CCL21 85-111 |
| SEQ ID NO: 10 | ASKTG KKGKGSKGCK RTERSQTPKG P | CCL21 86-111 |
| SEQ ID NO: 11 | SKTG KKGKGSKGCK RTERSQTPKG P | CCL21 87-111 |
| SEQ ID NO: 12 | KTG KKGKGSKGCK RTERSQTPKG P | CCL21 88-111 |
| SEQ ID NO: 13 | TG KKGKGSKGCK RTERSQTPKG P | CCL21 89-111 |
| SEQ ID NO: 14 | G KKGKGSKGCK RTERSQTPKG P | CCL21 90-111 |
| SEQ ID NO: 15 | KKGKGSKGCK RTERSQTPKG P | CCL21 91-111 |
| SEQ ID NO: 16 | KGKGSKGCK RTERSQTPKG P | CCL21 92-111 |
| SEQ ID NO: 17 | RKDRGASKTG KKGKGSKGCK RTERSQTPKG | CCL21 81-110 |
| SEQ ID NO: 18 | RKDRGASKTG KKGKGSKGCK RTERSQTPK | CCL21 81-109 |
| SEQ ID NO: 19 | RKDRGASKTG KKGKGSKGCK RTERSQTP | CCL21 81-108 |
| SEQ ID NO: 20 | RKDRGASKTG KKGKGSKGCK RTERSQT | CCL21 81-107 |
| SEQ ID NO: 21 | RKDRGASKTG KKGKGSKGCK RTERSQ | CCL21 81-106 |
| SEQ ID NO: 22 | RKDRGASKTG KKGKGSKGCK RTERS | CCL21 81-105 |
| SEQ ID NO: 23 | RKDRGASKTG KKGKGSKGCK RTER | CCL21 81-104 |
| SEQ ID NO: 24 | RKDRGASKTG KKGKGSKGCK RTE | CCL21 81-103 |
| SEQ ID NO: 25 | RKDRGASKTG KKGKGSKGCK RT | CCL21 81-102 |
| SEQ ID NO: 26 | RKDRGASKTG KKGKGSKGCK R | CCL21 81-101 |
| SEQ ID NO: 27 | RKDRGASKTG KKGKGSKGCK | CCL21 81-100 |
| SEQ ID NO: 28 | RKDRGASKTG KKGKGSKGC | CCL21 81-99 |
| SEQ ID NO: 29 | RKDRGASKTG KKGKGSKG | CCL21 81-98 |
| SEQ ID NO: 30 | RKDRGASKTG KKGKGSK | CCL21 81-97 |
| SEQ ID NO: 31 | RKDRGASKTG KKGKGS | CCL21 81-96 |
| SEQ ID NO: 32 | RKDRGASKTG KKGKG | CCL21 81-95 |
| SEQ ID NO: 33 | RKDRGASKTG KKGK | CCL21 81-94 |
| SEQ ID NO: 34 | MAQSLALSLLILVLAFGIPRTQGSDGGAQDCCLKYSQRKIPAKVVR | CCL21 1-134 w. |
| | SYRKQEPSLGCSIPAILFLPRKRSQAELCADPKELWVQQLMQHLD | signal peptide |
| | KTPSPQKPAQGCRKDRGASKTGKKGKGSKGCKRTERSQTPKGP | (underlined) |
| SEQ ID NO: 35 | SDGGAQDCCLKYSQRKIPAKVVRSYRKQEPSLGCSIPAILFLPRKR | CCL21 1-111 |
| | SQAELCADPKELWVQQLMQHLDKTPSPQKPAQGCRKDRGASKT | w/o signal |
| | GKKGKGSKGCKRTERSQTPKGP | peptide |
| SEQ ID NO: 36 | PSPQKPAQGC RKDRGASKTG K | CCL21 71-91 |

SEQ ID NO: 34 is the full length amino acid sequence of CCL21 including signal peptide 1-23 according to NCBI Reference Sequence: NP_002980.1.

SEQ ID NO: 35 is the full length amino acid sequence of CCL21 without the signal peptide. This is the CCL21 reference sequence all other CCL21 peptides of the present disclosure are numbered according to.

The peptides of the present disclosure comprise or consist of the C-terminal tail of human CCL21 (SEQ ID NO: 1) in addition to variants, fragments and variants of fragments thereof.

SEQ ID NO: 1 corresponds to full length C-terminal tail of CCL21, i.e. CCL21 71-111 which can be divided into four domains, D1-D4. D1 corresponds to amino acid residues 1-10 of SEQ ID NO: 1; D2 corresponds to amino acid residues 11-21 of SEQ ID NO: 1; D3 corresponds to amino acid residues 22-31 of SEQ ID NO: 1; and D4 corresponds to amino acid residues 32-41 of SEQ ID NO: 1.

SEQ ID NO: 2-4 contain certain of the domains: SEQ ID NO: 2 contains domains D1-D3, corresponding to amino acid residues 1-31 of SEQ ID NO: 1 and 71-101 of CCL21; SEQ ID NO: 3 contains domains D2-D3, corresponding to amino acid residues 11-31 of SEQ ID NO:1 and 81-101 of CCL21; and SEQ ID NO: 4 contains domains D2-D4, corresponding to amino acid residues 11-41 of SEQ ID NO: 1.

SEQ ID NO: 5 contains domains D2-D4, corresponding to amino acid residues 11-41 of SEQ ID NO: 1, and has further two amino acid substitutions corresponding to substitution of aspartic acid 13 with alanine (D13A) and glutamic acid 33 with alanine (E33A).

SEQ ID NO: 6-16 are N terminally truncated fragments of CCL21 71-111 (SEQ ID NO: 1).

SEQ ID NO: 36 is a C terminally truncated fragment of CCL21 71-111 (SEQ ID NO: 1).

SEQ ID NO: 17-33 are N and C terminally truncated fragments of CCL21 71-111 (SEQ ID NO: 1).

### DETAILED DESCRIPTION

The chemokine CCL21 coordinates the meeting between antigen presenting DCs and naïve T-cells. It has previously been suggested that direct intra-tumoral CCL21 injections could increase the number of tumor infiltrating T-cell (TILs) directed against antigens characterizing the otherwise poorly immunogenic tumors. CCL21 is, however, a relatively weak chemo-attractant.

It has been surprisingly found that the C-terminal tail of CCL21 and variants, fragments or variants of fragments thereof are capable of potentiating the chemotactic potential of CCL21 and reduce tumor growth in an in vivo tumor mouse model. The observed improvement of chemotactic potential of endogenous or supplemented CCL21 in the tumor should provide increase in the number of tumor infiltrating T-cell (TILs) directed against antigens characterizing the tumor.

This increased chemotactic potential of CCL21 can be used for exploitation of the immune system in anti-cancer therapies and maximizing immune system attack on the cancerous cells.

It will be clear for the person skilled in the art, that aspects and/or embodiments as described herein may be combined.

Chemotaxis-Potentiating Peptides

In one embodiment, an isolated polypeptide having a length of less than 100 amino acids comprising or consisting of an amino acid sequence according to SEQ ID NO: 1 or a fragment or variant thereof, wherein said polypeptide is capable of binding to glycosaminoglycans (GAGs) is provided.

In one embodiment, an isolated polypeptide having a length of less than 100 amino acids comprising or consisting of a variant of SEQ ID NO: 1, wherein said variant has at least 80%, but less than 99% sequence identity to SEQ ID NO: 1, wherein said polypeptide is capable of binding to glycosaminoglycans (GAGs) is provided.

In one embodiment, said variant has at least 85%, but less than 99% sequence identity to SEQ ID NO: 1.

In one embodiment, said variant has at least 90%, but less than 99% sequence identity to SEQ ID NO: 1.

In one embodiment, said variant has at least 95%, but less than 99% sequence identity to SEQ ID NO: 1.

In one embodiment, said variant has at least 97%, but less than 99% sequence identity to SEQ ID NO: 1.

In one embodiment, an isolated polypeptide having a length of less than 100 amino acids comprising or consisting of a variant of SEQ ID NO: 1, wherein said variant has between 1 and 10 amino acid substitutions as compared to SEQ ID NO: 1, wherein said polypeptide is capable of binding to glycosaminoglycans (GAGs) is provided.

In one embodiment, said variant has between 1 and 8 amino acid substitutions as compared to SEQ ID NO: 1.

In one embodiment, said variant has between 1 and 5 amino acid substitutions as compared to SEQ ID NO: 1.

In one embodiment, said variant has between 1 and 3 amino acid substitutions as compared to SEQ ID NO: 1.

In one embodiment, an isolated polypeptide having a length of less than 100 amino acids comprising or consisting of a fragment of SEQ ID NO: 1, or a variant thereof having between 1 and 10 amino acid substitutions as compared to SEQ ID NO: 1, wherein said polypeptide is capable of binding to glycosaminoglycans (GAGs) is provided.

In one embodiment, said variant has between 1 and 8 amino acid substitutions as compared to SEQ ID NO: 1.

In one embodiment, said variant has between 1 and 5 amino acid substitutions as compared to SEQ ID NO: 1.

In one embodiment, said variant has between 1 and 3 amino acid substitutions as compared to SEQ ID NO: 1, such as 1, 2 or 3.

In one embodiment, an isolated polypeptide having a length of less than 100 amino acids comprising or consisting of an amino acid sequence differing from SEQ ID NO: 1 by truncation at the N-terminus by at least one amino acid, such as between 1-20 amino acids, or a variant thereof having between 1 and 10 amino acid substitutions, such as 1-5 amino acid substitutions as compared to SEQ ID NO: 1, wherein said polypeptide is capable of binding to glycosaminoglycans (GAGs) is provided.

In one embodiment, said isolated polypeptide differs from SEQ ID NO: 1 by truncation at the N-terminus by between 1-20 amino acids.

In one embodiment, said isolated polypeptide differs from SEQ ID NO: 1 by truncation at the N-terminus by between 1-19 amino acids.

In one embodiment, said isolated polypeptide differs from SEQ ID NO: 1 by truncation at the N-terminus by between 1-18 amino acids.

In one embodiment, said isolated polypeptide differs from SEQ ID NO: 1 by truncation at the N-terminus by between 1-17 amino acids.

In one embodiment, said isolated polypeptide differs from SEQ ID NO: 1 by truncation at the N-terminus by between 1-16 amino acids.

In one embodiment, said isolated polypeptide differs from SEQ ID NO: 1 by truncation at the N-terminus by between 1-15 amino acids.

In one embodiment, said isolated polypeptide differs from SEQ ID NO: 1 by truncation at the N-terminus by between 1-10 amino acids.

In one embodiment, said isolated polypeptide differs from SEQ ID NO: 1 by truncation at the N-terminus by between 1-9 amino acids.

In one embodiment, said isolated polypeptide differs from SEQ ID NO: 1 by truncation at the N-terminus by between 1-8 amino acids.

In one embodiment, said isolated polypeptide differs from SEQ ID NO: 1 by truncation at the N-terminus by between 1-7 amino acids.

In one embodiment, said isolated polypeptide differs from SEQ ID NO: 1 by truncation at the N-terminus by between 1-6 amino acids.

In one embodiment, said isolated polypeptide differs from SEQ ID NO: 1 by truncation at the N-terminus by between 1-5 amino acids.

In one embodiment, said isolated polypeptide differs from SEQ ID NO: 1 by truncation at the N-terminus by between 1-4 amino acids.

In one embodiment, said isolated polypeptide differs from SEQ ID NO: 1 by truncation at the N-terminus by between 1-3 amino acids, such as 2 amino acids.

In one embodiment, said N-terminally truncated polypeptide has between 1 and 8 amino acid substitutions as compared to SEQ ID NO: 1.

In one embodiment, said N-terminally truncated polypeptide has between 1 and 5 amino acid substitutions as compared to SEQ ID NO: 1.

In one embodiment, said N-terminally truncated polypeptide has between 1 and 3 amino acid substitutions as compared to SEQ ID NO: 1, such as 1, 2 or 3.

In one embodiment, an isolated polypeptide having a length of less than 100 amino acids comprising or consisting of an amino acid sequence differing from SEQ ID NO: 1 by truncation at the C-terminus by at least one amino acid, such as between 1-19 amino acids, or a variant thereof having between 1 and 10 amino acid substitutions as compared to SEQ ID NO: 1. such as 1-5 amino acid substitutions, wherein said polypeptide is capable of binding to glycosaminoglycans (GAGs) is provided.

In one embodiment, said isolated polypeptide differs from SEQ ID NO: 1 by truncation at the C-terminus by between 1-18 amino acids.

In one embodiment, said isolated polypeptide differs from SEQ ID NO: 1 by truncation at the C-terminus by between 1-17 amino acids.

In one embodiment, said isolated polypeptide differs from SEQ ID NO: 1 by truncation at the C-terminus by between 1-16 amino acids.

In one embodiment, said isolated polypeptide differs from SEQ ID NO: 1 by truncation at the C-terminus by between 1-15 amino acids.

In one embodiment, said isolated polypeptide differs from SEQ ID NO: 1 by truncation at the C-terminus by between 1-14 amino acids.

In one embodiment, said isolated polypeptide differs from SEQ ID NO: 1 by truncation at the C-terminus by between 1-13 amino acids.

In one embodiment, said isolated polypeptide differs from SEQ ID NO: 1 by truncation at the C-terminus by between 1-12 amino acids.

In one embodiment, said isolated polypeptide differs from SEQ ID NO: 1 by truncation at the C-terminus by between 1-11 amino acids.

In one embodiment, said isolated polypeptide differs from SEQ ID NO: 1 by truncation at the C-terminus by between 1-10 amino acids.

In one embodiment, said isolated polypeptide differs from SEQ ID NO: 1 by truncation at the C-terminus by between 1-9 amino acids.

In one embodiment, said isolated polypeptide differs from SEQ ID NO: 1 by truncation at the C-terminus by between 1-8 amino acids.

In one embodiment, said isolated polypeptide differs from SEQ ID NO: 1 by truncation at the C-terminus by between 1-7 amino acids.

In one embodiment, said isolated polypeptide differs from SEQ ID NO: 1 by truncation at the C-terminus by between 1-6 amino acids.

In one embodiment, said isolated polypeptide differs from SEQ ID NO: 1 by truncation at the C-terminus by between 1-5 amino acids.

In one embodiment, said isolated polypeptide differs from SEQ ID NO: 1 by truncation at the C-terminus by between 1-4 amino acids.

In one embodiment, said isolated polypeptide differs from SEQ ID NO: 1 by truncation at the C-terminus by between 1-3 amino acids, such as 2 amino acids.

In one embodiment, said C-terminally truncated polypeptide has between 1 and 8 amino acid substitutions as compared to SEQ ID NO: 1.

In one embodiment, said C-terminally truncated polypeptide has between 1 and 5 amino acid substitutions as compared to SEQ ID NO: 1.

In one embodiment, said C-terminally truncated polypeptide has between 1 and 3 amino acid substitutions as compared to SEQ ID NO: 1, such as 1, 2 or 3.

In one embodiment, the polypeptide has an amino acid sequence differing from SEQ ID NO: 1 by truncation at the N-terminus and the C terminus by one or more amino acids or a variant thereof. The polypeptide may be N-terminally truncated by at least one amino acid, such as between 1-20 amino acids, such as between 1-15 amino acids, for example between 1-10 amino acids, such as between 1-5 amino acids. The polypeptide may be truncated at the C-terminus by at least one amino acid, such as between 1-19 amino acids, such as between 1-15 amino acids, for example between 1-10 amino acids, such as between 1-5 amino acids. In one embodiment, the polypeptide is truncated from both the N terminal and C terminal end by between 1-15 amino acids. The polypeptide may also be a variant of the N and C terminally truncated peptide having between 1 and 5 amino acid substitutions relative to SEQ ID NO: 1, such as 1, 2 or 3 amino acid substitutions relative to SEQ ID NO: 1.

In one embodiment, the fragment of SEQ ID NO: 1, which may be an N terminally truncated and/C terminally truncated version of SEQ ID NO: 1, has a length of at least 10 amino acids, such as at least 11, 12, 13, 14, 15, 16, 17, 18, 19 or 20 amino acids or is a variant thereof having between 1-5 amino acid substitutions relative to said sequence, such as 1, 2 or 3 amino acid substitutions. In one embodiment the fragment is at least 15 amino acids.

In one embodiment, the polypeptide consists of an amino acid sequence according to any one of SEQ ID NOs: 1-33, or a variant thereof having at least 80% sequence identity, such as at least 90%, such as at least 95% sequence identity to said sequence and having at least one amino acid substitution relative thereto, such as 1, 2 or 3 amino acid substitutions.

In one embodiment, the polypeptide consists of an amino acid sequence according to any of SEQ ID NOs: 2, 3, or 6-33 or a variant of any of said sequences, wherein said variant has at least 80% sequence identity to said sequence, such as at least 90%, such as at least 95% sequence identity to said sequence, and wherein said sequence comprises at least one amino acid substitution relative to said sequence, such as 1, 2 or 3 amino acid substitutions.

In one embodiment, the polypeptide comprises or consists of CCL21 89-111 (SEQ ID NO: 13) or is a variant thereof having between 1 and 10 amino acid substitutions as compared to SEQ ID NO: 1, such as 1, 2 or 3 amino acid substitutions.

In one embodiment, the polypeptide comprises or consists of CCL21 82-111 (SEQ ID NO: 6) or is a variant thereof having between 1 and 10 amino acid substitutions as compared to SEQ ID NO: 1, such as 1, 2 or 3 amino acid substitutions.

In one embodiment, the polypeptide comprises or consists of an amino acid sequence according to SEQ ID NO: 1, or a variant thereof having between 1 and 10 amino acid substitutions as compared to SEQ ID NO: 1.

In one embodiment, the polypeptide comprises or consists of an amino acid sequence according to positions 1-31 of SEQ ID NO: 1, corresponding to SEQ ID NO: 2, or a variant thereof having between 1 and 10 amino acid substitutions as compared to SEQ ID NO: 1.

In one embodiment, the polypeptide comprises or consists of a variant of SEQ ID NO: 2 having between 1 and 8 amino acid substitutions as compared to SEQ ID NO: 1.

In one embodiment, the polypeptide comprises or consists of a variant of SEQ ID NO: 2 having between 1 and 5 amino acid substitutions as compared to SEQ ID NO: 1.

In one embodiment, the polypeptide comprises or consists of a variant of SEQ ID NO: 2 having between 1 and 3 amino acid substitutions as compared to SEQ ID NO: 1.

In one embodiment, the polypeptide comprises or consists of an amino acid sequence according to positions 11-31 of SEQ ID NO: 1, corresponding to SEQ ID NO: 3, or a variant thereof having between 1 and 10 amino acid substitutions as compared to SEQ ID NO: 1.

In one embodiment, the polypeptide comprise or consist of a variant of SEQ ID NO: 3 having between 1 and 8 amino acid substitutions as compared to SEQ ID NO: 1.

In one embodiment, the polypeptide comprise or consist of a variant of SEQ ID NO: 3 having between 1 and 5 amino acid substitutions as compared to SEQ ID NO: 1.

In one embodiment, the polypeptide comprise or consist of a variant of SEQ ID NO: 3 having between 1 and 3 amino acid substitutions as compared to SEQ ID NO: 1.

In one embodiment, the polypeptide comprises or consists of an amino acid sequence according to positions 11-41 of SEQ ID NO: 1 (and positions 81-111 of SEQ ID NO: 35 (CCL21)), corresponding to SEQ ID NO: 4 (CCL21 81-111), or a variant thereof having between 1 and 10 amino acid substitutions as compared to SEQ ID NO: 1.

In one embodiment, the polypeptide comprises or consists of a variant of SEQ ID NO: 4 having between 1 and 8 amino acid substitutions as compared to SEQ ID NO: 1. In one embodiment, the polypeptide comprises or consists of a variant of SEQ ID NO: 4 having between 1 and 5 amino acid substitutions as compared to SEQ ID NO: 1.

In one embodiment, the polypeptide comprises or consists of a variant of SEQ ID NO:

4 having between 1 and 3 amino acid substitutions as compared to SEQ ID NO: 1.

In one embodiment, the polypeptide comprises or consists of an amino acid sequence according to positions 82-111 of SEQ ID NO: 35 (CCL21), corresponding to SEQ ID NO: 6, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 83-111 of SEQ ID NO: 35 (CCL21), corresponding to SEQ ID NO: 7, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 84-111 of SEQ ID NO: 35 (CCL21), corresponding to SEQ ID NO: 8, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 85-111 of SEQ ID NO: 35 (CCL21), corresponding to SEQ ID NO: 9, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 86-111 of SEQ ID NO: 35 (CCL21), corresponding to SEQ ID NO: 10, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 87-111 of SEQ ID NO: 35 (CCL21), corresponding to SEQ ID NO: 11, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 88-111 of SEQ ID NO: 35 (CCL21), corresponding to SEQ ID NO: 12, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 89-111 of SEQ ID NO: 35 (CCL21), corresponding to SEQ ID NO: 13, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 90-111 of SEQ ID NO: 35 (CCL21), corresponding to SEQ ID NO: 14, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 91-111 of SEQ ID NO: 35 (CCL21), corresponding to SEQ ID NO: 15, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 92-111 of SEQ ID NO: 35 (CCL21), corresponding to SEQ ID NO: 16, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 81-110 of SEQ ID NO: 35 (CCL21), corresponding to SEQ ID NO: 17, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 81-109 of SEQ ID NO: 35 (CCL21), corresponding to SEQ ID NO: 18, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 81-108 of SEQ ID NO: 35 (CCL21), corresponding to SEQ ID NO: 19, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 81-107 of SEQ ID NO: 35 (CCL21), corresponding to SEQ ID NO: 20, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 81-106 of SEQ ID NO: 35 (CCL21), corresponding to SEQ ID NO: 21, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 81-105 of SEQ ID NO: 35 (CCL21), corresponding to SEQ ID NO: 22, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 81-104 of SEQ ID NO: 35 (CCL21), corresponding to SEQ ID NO: 23, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 81-103 of SEQ ID NO: 35 (CCL21), corresponding to SEQ ID NO: 24, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 81-102 of SEQ ID NO: 35 (CCL21), corresponding to SEQ ID NO: 25, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 81-101 of SEQ ID NO: 35 (CCL21), corresponding to SEQ ID NO: 26, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 81-100 of SEQ ID NO: 35 (CCL21), corresponding to SEQ ID NO: 27, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 81-99 of SEQ ID NO: 35 (CCL21), corresponding to SEQ ID NO: 28, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 81-98 of SEQ ID NO: 35 (CCL21), corresponding to SEQ ID NO: 29, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 81-97 of SEQ ID NO: 35 (CCL21), corresponding to SEQ ID NO: 30, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 81-96 of SEQ ID NO: 35 (CCL21), corresponding to SEQ ID NO: 31, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 81-95 of SEQ ID NO: 35 (CCL21), corresponding to SEQ ID NO: 32, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide comprises or consists of an amino acid sequence according to positions 81-94 of SEQ ID NO: 35 (CCL21), corresponding to SEQ ID NO: 33, or a variant thereof having between 1 and 10 amino acid substitutions as compared to SEQ ID NO: 1, such as between 1 and 5 amino acid substitutions, such as 1, 2 or 3 amino acid substitutions as compared to SEQ ID NO: 1.

In one embodiment, the polypeptide has a length of less than 90, 85, 80, 75, 70, 65, 60, 55, 50, 45, 40, 35, 30, 25, 20, 15 or 10 amino acids.

In one embodiment, the polypeptide has a length of less than 90 amino acids.

In one embodiment, the polypeptide has a length of less than 85 amino acids.

In one embodiment, the polypeptide has a length of less than 80 amino acids.

In one embodiment, the polypeptide has a length of less than 75 amino acids.

In one embodiment, the polypeptide has a length of less than 70 amino acids.

In one embodiment, the polypeptide has a length of less than 65 amino acids.

In one embodiment, the polypeptide has a length of less than 60 amino acids.

In one embodiment, the polypeptide has a length of less than 55 amino acids.

In one embodiment, the polypeptide has a length of less than 50 amino acids.

In one embodiment, the polypeptide has a length of less than 45 amino acids.

In one embodiment, the polypeptide has a length of less than 40 amino acids.

In one embodiment, the polypeptide has a length of less than 35 amino acids.

In one embodiment, the polypeptide has a length of less than 30 amino acids.

In one embodiment, the polypeptide has a length of less than 25 amino acids.

In one embodiment, the polypeptide has a length of less than 20 amino acids.

In one embodiment, the polypeptide has a length of less than 15 amino acids.

In one embodiment, the polypeptide has a length of less than 10 amino acids.

In one embodiment the polypeptide has a length of at least 10 amino acids, such at least 11, 12, 13, 14, 15, 16, 17, 18, 19, 20, 21, 22, 23, 24, 25, 26, 27, 28, 29 or 30 amino acids.

In one embodiment the polypeptide has a length of at least 14 amino acids.

In one embodiment the polypeptide has a length of at least 15 amino acids.

In one embodiment the polypeptide has a length of at least 20 amino acids.

In one embodiment, the polypeptide comprises or consists of a variant of SEQ ID NO: 1 or a variant of a fragment of SEQ ID NO: 1 as described herein, wherein said variant has at least 90% sequence identity to SEQ ID NO: 1, such as at least 95% sequence identity to SEQ ID NO: 1, for example at least 97% sequence identity to SEQ ID NO: 1.

In one embodiment, said variant has at least 90% sequence identity to SEQ ID NO: 1.

In one embodiment, said variant has at least 95% sequence identity to SEQ ID NO: 1.

In one embodiment, said variant has at least 97% sequence identity to SEQ ID NO: 1.

In one embodiment, the polypeptide comprises or consists of a variant of SEQ ID NO: 1 or a variant of a fragment of SEQ ID NO: 1 as described herein, wherein the variant has between 1 and 5 amino acid substitutions as compared to SEQ ID NO: 1.

In one embodiment, the polypeptide comprises or consists of a variant of SEQ ID NO: 1 or a variant of a fragment of SEQ ID NO: 1 as described herein, wherein the variant has between 1 and 3 amino acid substitutions as compared to SEQ ID NO: 1, such as 1, 2 or 3 amino acid substitutions.

In one embodiment, the amino acid substitutions are conservative substitutions.

In one embodiment, the amino acid substitutions increase the net charge of said polypeptide.

In one embodiment, the amino acid substitutions are substitution of negative or neutral amino acid residues to positive amino acid residues.

In one embodiment, the amino acid substitutions are substitution of negative amino acid residues to neutral or positive amino acid residues.

In one embodiment, the amino acid substitutions comprise a D to A substitution at position 13 of SEQ ID NO: 1 and/or an E to A substitution at position 33 of SEQ ID NO: 1.

In one embodiment, the polypeptide comprises or consists of an amino acid sequence according to SEQ ID NO: 5.

In one embodiment, the polypeptide further comprises one or more moieties conjugated to said polypeptide.

In one embodiment, the polypeptide as described herein further comprises one or more moieties conjugated to said polypeptide, wherein the one or more moieties are selected from the group consisting of albumin, fatty acids, polyethylene glycol (PEG), acylation groups, antibodies and antibody fragments. Such conjugation can result in an increased half-life of said polypeptide in vivo.

In one embodiment, the one or more moieties are selected from the group consisting of proteins, peptides or receptor ligands, wherein the one or more moieties are capable of directing delivery of the polypeptide to the site of the tumor.

In one embodiment, the polypeptide and the one or more moieties are conjugated to each other by a linker.

In one embodiment, an isolated polynucleotide encoding a polypeptide disclosed herein is provided.

In one embodiment, a vector comprising a polynucleotide encoding a polypeptide as disclosed herein is provided.

In one embodiment, said vector is a viral vector. Examples of viral vectors include but are not limited to adenoviruses, lentiviruses, adeno-associated viruses, herpesviruses, vaccinia viruses, poxviruses and oncolytic viruses. Said vector is suitable for expression of an isolated polypeptide as disclosed herein in a subject.

In one embodiment, said vector is an expression vector. Examples of expression vectors include, but are not limited to, E. coli expression vectors and SF9-insect expression vectors. Said expression vector is suitable for expression of an isolated polypeptide as disclosed herein in vitro.

In one embodiment, a host cell comprising a polynucleotide and/or comprising a vector as disclosed herein is provided.

In one embodiment, a pharmaceutical composition comprising an isolated polypeptide, an isolated polynucleotide encoding said polypeptide, a vector comprising a polynucleotide encoding said polypeptide or a host cell comprising a polynucleotide and/or comprising a vector comprising a polynucleotide encoding a polypeptide as disclosed herein is provided.

In one embodiment, said pharmaceutical composition is provided, wherein the polypeptide, the isolated polynucleotide, the vector or the host cell is formulated in a nanoparticle.

The pharmaceutical composition may comprise a pharmaceutically acceptable buffer, diluent, carrier, adjuvant and/or excipient and/or other customary pharmaceutical auxiliaries.

Treatment of Cancer

In one embodiment, the present disclosure relates to a method of treatment of cancer comprising administering to an individual in need thereof a therapeutically effective amount of an isolated polypeptide as disclosed herein.

In one embodiment, a method of potentiating immune activation in a tumor, the method comprising administering to an individual in need thereof a therapeutically effective amount of an isolated polypeptide as disclosed herein is provided.

In one embodiment, a method of potentiating the chemotactic potential of CCL21, the method comprising administering to an individual in need thereof a therapeutically effective amount of an isolated polypeptide as disclosed herein is provided.

In one embodiment, a method of treatment of cancer, potentiating immune activation in a tumor or potentiating the chemotactic potential of CCL21 is provided, wherein an isolated polypeptide as disclosed herein is administered in combination with an anti-cancer agent. Examples of anti-cancer agents include but are not limited to cyclophosphamide, Paclitaxel, 5-fluorouracil, CTLA-4 antagonists, PD-L1 antagonists or PD-1 antagonists.

In one embodiment, a method of treatment of cancer, potentiating immune activation in a tumor or potentiating the chemotactic potential of CCL21 is provided, wherein an isolated polypeptide as disclosed herein is administered in combination with radiation therapy.

In one embodiment, a method of treatment of cancer, potentiating immune activation in a tumor or potentiating the chemotactic potential of CCL21 is provided, wherein an isolated polypeptide as disclosed herein is administered in combination with cell-based anticancer immunotherapy.

In one embodiment, a method of treatment of cancer, potentiating immune activation in a tumor or potentiating the chemotactic potential of CCL21 is provided, wherein an isolated polypeptide as disclosed herein is administered in combination with a cytokine. Examples of cytokines include but are not limited to CCL21, INF-γ, IL-2, CXCL9, CXCL10 or CXCL12.

In one embodiment, a method of treatment of cancer, potentiating immune activation in a tumor or potentiating the chemotactic potential of CCL21 is provided, wherein an isolated polypeptide as disclosed herein is administered in combination with CCL21.

In one embodiment, a method of treatment of cancer, potentiating immune activation in a tumor or potentiating the chemotactic potential of CCL21 is provided, wherein the cancer is a CCR7-positive cancer.

In one embodiment, the present disclosure relates to a polypeptide as disclosed herein for use in the treatment of cancer.

In one embodiment, the present disclosure relates to a polypeptide as disclosed herein for use in potentiating immune activation in a tumor.

In one embodiment, the present disclosure relates to a polypeptide as disclosed herein for use in potentiating the chemotactic potential of CCL21.

In one embodiment, the present disclosure relates to a polypeptide as disclosed herein for use in the manufacture of a medicament for the treatment of cancer.

Items
1. A method of treatment of cancer comprising administering to an individual in need thereof a therapeutically effective amount of an isolated polypeptide having a length of less than 100 amino acids comprising or consisting of an amino acid sequence selected from the group consisting of
   a) the amino acid sequence according to SEQ ID NO: 1,
   b) a variant of SEQ ID NO: 1, wherein said variant has at least 80%, but less than 99% sequence identity to SEQ ID NO: 1;
   c) a variant of SEQ ID NO: 1, wherein said variant has between 1 and 10 amino acid substitutions as compared to SEQ ID NO: 1;
   d) a fragment of SEQ ID NO: 1, or a variant thereof having between 1 and 10 amino acid substitutions as compared to SEQ ID NO: 1;
   e) an amino acid sequence differing from SEQ ID NO: 1 by truncation at the N-terminus by at least one amino acid, such as between 1-10 amino acids, for example between 1-5 amino acids, or a variant thereof having between 1 and 10 amino acid substitutions as compared to SEQ ID NO: 1; or
   f) an amino acid sequence differing from SEQ ID NO: 1 by truncation at the C-terminus by at least one amino acid, such as between 1-10 amino acids, for example between 1-5 amino acids, or a variant thereof having between 1 and 10 amino acid substitutions as compared to SEQ ID NO: 1,
   wherein said polypeptide is capable of binding to glycosaminoglycans (GAGs).
2. A method of potentiating immune activation in a tumor, the method comprising administering to an individual in need thereof a therapeutically effective amount of an isolated polypeptide having a length of less than 100 amino acids comprising or consisting of an amino acid sequence selected from the group consisting of
   a) the amino acid sequence according to SEQ ID NO: 1,
   b) a variant of SEQ ID NO: 1, wherein said variant has at least 80%, but less than 99% sequence identity to SEQ ID NO: 1;
   c) a variant of SEQ ID NO: 1, wherein said variant has between 1 and 10 amino acid substitutions as compared to SEQ ID NO: 1;
   d) a fragment of SEQ ID NO: 1, or a variant thereof having between 1 and 10 amino acid substitutions as compared to SEQ ID NO: 1;
   e) an amino acid sequence differing from SEQ ID NO: 1 by truncation at the N-terminus by at least one amino acid, such as between 1-10 amino acids, for example between 1-5 amino acids, or a variant thereof having between 1 and 10 amino acid substitutions as compared to SEQ ID NO: 1; or
   f) an amino acid sequence differing from SEQ ID NO: 1 by truncation at the C-terminus by at least one amino acid, such as between 1-10 amino acids, for example between 1-5 amino acids, or a variant thereof having between 1 and 10 amino acid substitutions as compared to SEQ ID NO: 1,
   wherein said polypeptide is capable of binding to glycosaminoglycans (GAGs).
3. A method of potentiating the chemotactic potential of CCL21, the method comprising administering to an individual in need thereof a therapeutically effective amount of an isolated polypeptide having a length of less than 100 amino acids comprising or consisting of an amino acid sequence selected from the group consisting of
   a) the amino acid sequence according to SEQ ID NO: 1,
   b) a variant of SEQ ID NO: 1, wherein said variant has at least 80%, but less than 99% sequence identity to SEQ ID NO: 1;
   c) a variant of SEQ ID NO: 1, wherein said variant has between 1 and 10 amino acid substitutions as compared to SEQ ID NO: 1;
   d) a fragment of SEQ ID NO: 1, or a variant thereof having between 1 and 10 amino acid substitutions as compared to SEQ ID NO: 1;
   e) an amino acid sequence differing from SEQ ID NO: 1 by truncation at the N-terminus by at least one amino acid, such as between 1-10 amino acids, for example between 1-5 amino acids, or a variant thereof having between 1 and 10 amino acid substitutions as compared to SEQ ID NO: 1; or
   f) an amino acid sequence differing from SEQ ID NO: 1 by truncation at the C-terminus by at least one amino acid, such as between 1-10 amino acids, for example between 1-5 amino acids, or a variant thereof having between 1 and 10 amino acid substitutions as compared to SEQ ID NO: 1;
   wherein said polypeptide is capable of binding to glycosaminoglycans (GAGs).
4. The method according to any of the preceding items, wherein said polypeptide comprises or consists of an amino acid sequence according to positions 1-31 of SEQ ID NO: 1, corresponding to SEQ ID NO: 2, or a variant thereof having between 1 and 10 amino acid substitutions as compared to SEQ ID NO: 1.
5. The method according to any of the preceding items, wherein said polypeptide comprises or consists of an amino acid sequence according to positions 11-31 of SEQ ID NO: 1, corresponding to SEQ ID NO: 3, or a variant thereof having between 1 and 10 amino acid substitutions as compared to SEQ ID NO: 1.
6. The method according to any of the preceding items, wherein said polypeptide comprises or consists of an amino acid sequence according to positions 11-41 of SEQ ID NO: 1, corresponding to SEQ ID NO: 4, or a variant thereof having between 1 and 10 amino acid substitutions as compared to SEQ ID NO: 1.
7. The method according to any of the preceding items, wherein said polypeptide has a length of less than 90, 85, 80, 75, 70, 65, 60, 55, 50, 45, 40, 35, 30, 25, 20, 15 or 10 amino acids.
8. The method according to any of the preceding items, wherein said variant has at least 90% sequence identity to SEQ ID NO: 1, such as at least 95% sequence identity to SEQ ID NO: 1, for example at least 97% sequence identity to SEQ ID NO: 1.
9. The method according to any of the preceding items, wherein the variant has between 1 and 5 amino acid substitutions as compared to SEQ ID NO: 1.
10. The method according to any of the preceding items, wherein the variant has between 1 and 3 amino acid substitutions as compared to SEQ ID NO: 1.
11. The method according to any of the preceding items, wherein the amino acid substitutions are conservative substitutions.
12. The method according to any of the preceding items, wherein the amino acid substitutions increase the net charge of said polypeptide.
13. The method according to any of the preceding items, wherein the amino acid substitutions are substitutions of negative or neutral amino acid residues to positive amino acid residues.
14. The method according to any of the preceding items, wherein the amino acid substitutions are substitutions of negative amino acid residues to neutral or positive amino acid residues.
15. The method according to any of the preceding items, wherein said polypeptide comprises or consists of an amino acid sequence according to SEQ ID NO: 5 16. The method according to any of the preceding items, wherein said polypeptide further comprises one or more moieties conjugated to said polypeptide. 17. The method according to item 16, wherein the one or more moieties are selected from the group consisting of albumin, fatty acids, polyethylene glycol (PEG), acylation groups, antibodies and antibody fragments.
18. The method according to item 16, wherein the one or more moieties are selected from the group consisting proteins, peptides and receptor ligands.
19. The method according to any of items 16-18, wherein said polypeptide and the one or more moieties are conjugated to each other by a linker.
20. The method according to any of the preceding items, wherein said polypeptide is administered in combination with an anti-cancer agent, such as for example cyclophosphamide, Paclitaxel, 5-fluorouracil, CTLA-4 antagonists, PD-L1 antagonists or PD-1 antagonists.
21. The method according to any of the preceding items, wherein said polypeptide is administered in combination with radiation therapy.
22. The method according to any of the preceding items, wherein said polypeptide is administered in combination with cell-based anticancer immunotherapy.
23. The method according to any of the preceding items, wherein said polypeptide is administered in combination with a cytokine, such as for example CCL21, INF-γ, IL-2, CXCL9, CXCL10 or CXCL12.
24. The method according to any of the preceding items, wherein said polypeptide is administered in combination with CCL21.
25. The method according to any of the preceding items, wherein the cancer is a CCR7-positive cancer.
26. A polypeptide for use in the treatment of cancer or for potentiating immune activation in a tumor or for potentiating the chemotactic potential of CCL21 having a length of less than 100 amino acids comprising or consisting of an amino acid sequence selected from the group consisting of
   a) the amino acid sequence according to SEQ ID NO: 1,
   b) a variant of SEQ ID NO: 1, wherein said variant has at least 80%, but less than 99% sequence identity to SEQ ID NO: 1;
   c) a variant of SEQ ID NO: 1, wherein said variant has between 1 and 10 amino acid substitutions as compared to SEQ ID NO: 1;
   d) a fragment of SEQ ID NO: 1, or a variant thereof having between 1 and 10 amino acid substitutions as compared to SEQ ID NO: 1;
   e) an amino acid sequence differing from SEQ ID NO: 1 by truncation at the N-terminus by at least one amino acid, such as between 1-10 amino acids, for example between 1-5 amino acids, or a variant thereof having between 1 and 10 amino acid substitutions as compared to SEQ ID NO: 1; or
   f) an amino acid sequence differing from SEQ ID NO: 1 by truncation at the C-terminus by at least one amino acid, such as between 1-10 amino acids, for example between 1-5 amino acids, or a variant thereof having between 1 and 10 amino acid substitutions as compared to SEQ ID NO: 1;
   wherein said polypeptide is capable of binding to glycosaminoglycans (GAGs).
27. An isolated polypeptide having a length of less than 100 amino acids comprising or consisting of an amino acid sequence selected from the group consisting of
   a) the amino acid sequence according to SEQ ID NO: 1,
   b) a variant of SEQ ID NO: 1, wherein said variant has at least 80%, but less than 99% sequence identity to SEQ ID NO: 1;
   c) a variant of SEQ ID NO: 1, wherein said variant has between 1 and 10 amino acid substitutions as compared to SEQ ID NO: 1;
   d) a fragment of SEQ ID NO: 1, or a variant thereof having between 1 and 10 amino acid substitutions as compared to SEQ ID NO: 1;
   e) an amino acid sequence differing from SEQ ID NO: 1 by truncation at the N-terminus by at least one amino acid, such as between 1-10 amino acids, for example between 1-5 amino acids, or a variant thereof having between 1 and 10 amino acid substitutions as compared to SEQ ID NO: 1; or
   f) an amino acid sequence differing from SEQ ID NO: 1 by truncation at the C-terminus by at least one amino acid, such as between 1-10 amino acids, for example between 1-5 amino acids, or a variant thereof having between 1 and 10 amino acid substitutions as compared to SEQ ID NO: 1,
   wherein said polypeptide is capable of binding to glycosaminoglycans (GAGs).
28. The polypeptide according to item 27, wherein said polypeptide comprises or consists of an amino acid sequence according to positions 1-31 of SEQ ID NO: 1, corresponding to SEQ ID NO: 2, or a variant thereof having between 1 and 10 amino acid substitutions as compared to SEQ ID NO: 1.
29. The polypeptide according to item 27, wherein said polypeptide comprises or consists of an amino acid sequence according to positions 11-31 of SEQ ID NO: 1, corresponding to SEQ ID NO: 3, or a variant thereof having between 1 and 10 amino acid substitutions as compared to SEQ ID NO: 1.
30. The polypeptide according to item 27, wherein said polypeptide comprises or consists of an amino acid sequence according to positions 11-41 of SEQ ID NO: 1, corresponding to SEQ ID NO: 4, or a variant thereof having between 1 and 10 amino acid substitutions as compared to SEQ ID NO: 1.
31. The polypeptide according to any of items 27 to 30, wherein said polypeptide has a length of less than 90, 85, 80, 75, 70, 65, 60, 55, 50, 45, 40, 35, 30, 25, 20, 15 or 10 amino acids.
32. The polypeptide according to any of items 27 to 31, wherein said variant has at least 90% sequence identity to SEQ ID NO: 1, such as at least 95% sequence identity to SEQ ID NO: 1, for example at least 97% sequence identity to SEQ ID NO: 1.
33. The polypeptide according to any of items 27 to 32, wherein the variant has between 1 and 5 amino acid substitutions as compared to SEQ ID NO: 1.
34. The polypeptide according to any of items 27 to 33, wherein the variant has between 1 and 3 amino acid substitutions as compared to SEQ ID NO: 1.
35. The polypeptide according to any of items 27 to 34, wherein the amino acid substitutions are conservative substitutions.
36. The polypeptide according to any of items 27 to 35, wherein the amino acid substitutions increase the net charge of said polypeptide.
37. The polypeptide according to any of items 27 to 36, wherein the amino acid substitutions are substitution of negative or neutral amino acid residues to positive amino acid residues.
38. The polypeptide according to any of items 27 to 37, wherein the amino acid substitutions are substitution of negative amino acid residues to neutral or positive amino acid residues.
39. The polypeptide according to any of items 27 to 38, wherein said polypeptide comprises or consists of an amino acid sequence according to SEQ ID NO: 5 40. The polypeptide according to any of items 27 to 39, wherein said polypeptide further comprises one or more moieties conjugated to said polypeptide.
41. The polypeptide according to item 40, wherein the one or more moieties are selected from the group consisting of albumin, fatty acids, polyethylene glycol (PEG), acylation groups, antibodies and antibody fragments.
42. The method according to item 40, wherein the one or more moieties are selected from the group consisting proteins, peptides and receptor ligands.
43. The polypeptide according to any of items 40-42, wherein said polypeptide and the one or more moieties are conjugated to each other by a linker.
44. An isolated polynucleotide encoding the polypeptide according to any of items 27-43.
45. A vector comprising the polynucleotide according to item 44.
46. The vector according to item 45, wherein the vector is a viral vector, selected from the group consisting of adenoviruses, lentiviruses, adeno-associated viruses, herpesviruses, vaccinia viruses, poxviruses and oncolytic viruses.
47. The vector according to item 45, wherein the vector is an expression vector, such as an expression vector selected from the group consisting of E. coli expression vector and SF9-insect expression vectors.
48. A host cell comprising the polynucleotide according to item 44 and/or the vector according to any of items 45 and 46.
49. A pharmaceutical composition comprising the polypeptide according to any of items 27-43, the isolated polynucleotide according to item 44, the vector according to any of items 45-46 or the host cell according to item 48.
50. The pharmaceutical composition according to item 49, wherein the polypeptide according to any of items 27-43, the isolated polynucleotide according to item 44, the vector according to any of items 45-46 or the host cell according to item 48 is formulated in a nanoparticle.
51. The isolated polypeptide according to any of items 27-43, the isolated polynucleotide according to item 44, the vector according to any of items 45-46 or the host cell according to item 48 for use as a medicament.
52. The isolated polypeptide according to any of items 27-43, the isolated polynucleotide according to item 44, the vector according to any of items 45-46 or the host cell according to item 48 for use in the manufacture of a medicament for treatment of cancer.

### EXAMPLES

### Example 1, Effect of Peptides on Chemotaxis of Monocyte Derived Human DCs (moDC)

Ibidi3D μ-slide chemotaxis assay. Mature monocyte derived human DCs were thawed by adding pre-warmed X-vivo, 2% FCS and Glutamine, dissolving in a total of 10 ml of medium. After centrifugation (220×g, 5 min), the DCs were resuspended in fresh medium and left to acclimatize for 20 min.

The collagen mixture with cells was prepared according to the manufacturer's protocol. Briefly, 10 μl 7.5% NaHCO₃ was mixed with 20 μl 10×MEM and 150 μl Pure Col was added. After mixing, 90 μl of a mixture of DCs (1×10\hat{ }6 cells/ml) and CCL21 Tail peptide (final concentration 10 μM) was added. The cell/collagen solution was transferred to the ibidi μ-slide channel holding 6 μl. The collagen with cells was allowed to crosslink in the incubator for 45 minutes in a humidified petridish (holding napkins wetted in sterile water). While filling sink and source reservoirs, the channel and second reservoir were kept plugged. Medium with chemokine buffer (X-vivo 15, 2% HS, glutamine) and Tail-peptide was added to the sink (60 μl), and medium with chemokine plus Tail-peptide was added to the source (60 μl). The setup was transferred to Time-lapse. Cells were tracked for 10 hours and films were analyzed using Autozell tracking program. A population based chemotactic index (CI) was extracted using MatLab.

It was found that SEQ ID NO: 1 potentiates chemotaxis of moDC induced by 10 nM CCL21 in a dose dependent manner, up to 26 fold (FIG. 1, column 1-4). In contrast, it was also found that SEQ ID NO: 1 potentiates the effect of CCL19 by less than two fold (FIG. 1, column 5-8), and there was no strong chemotaxis signal potentiated by CXCL12gamma-induced migration (FIG. 1, column 9-12).

The effect of SEQ ID NO: 1 on moDC chemotaxis was reflected by the spidergrams extracted during chemotaxis analysis (FIG. 2A) as well as being readily observable by the change in moDC shape/morphology during migration (FIG. 2B-D).

It was found that SEQ ID NO: 4 and 5 were able to potentiate CCL21 induced chemotaxis of moDC with similar potential as SEQ ID NO: 1 (FIG. 3).

### Example 2, Effect of Peptides on CCR7 Signalling Via Gαi

CCR7 signalling induced by CCL19, CCL21, CCL19 chimera (CCL19 with CCL21 tail) and tailless CCL21 was investigated. Signalling was detected as a decrease in cAMP which is reflected by an increase in BRET (Bioluminescence resonance energy transfer) ratio.

CHO cells were grown in RPMI medium with 10% FBS and 1% Penicillin/Streptomycin. Cells where seeded in a concentration yielding 1,000,000 cells/well on transfection day (using 6-well-plates).

Cells were transfected with CCR7 and Camyel-encoding plasmids (in 1:5 ratio) using lipofectamine with a total of 1 pg of DNA and 6 pg lipofectamine per well. After 24 hours, the cells where washed in PBS and resuspended in PBS with glucose (final concentration 5 mM).

After harvesting, cells where pooled in two groups. One group was used as a control, whereas the other was supplemented with SEQ ID NO: 1 (CCL21 tail peptide (D1-4)) in a final concentration of 10 μM.

The cells where plated in a 96-well black-white iso plate according to setup. Coelenterazine (final concentration of 5 μM) was added and cells were incubated for 10 min. The 4 ligands (CCL19, CCL21, CCL19 chimera, and tailless CCL21) were added to respective wells. An equal amount chemokine buffer was added to wells without ligands. Forskolin was added to the wells 5 min after addition of ligands.

The BRET ratio was measured as the ratio eYFP/RLuc (BRET 525 emission/BRET 485 emission). In the absence of ligand, bioluminescence resonance energy transfer (BRET) between RLuc and eYFP, both harboured by the Camyel sensor, is high, giving rise to emission from both RLuc and eYFP and a concomitant high BRET ratio. When cAMP binds to the camyel sensor, bioluminescence resonance energy transfer from RLuc to eYFP is decreased, lowering emission from eYFP and thus lowering the BRET ratio. This means that stimulation of receptors that signal via an increase in cAMP leads to a decrease in BRET ratio. The opposite is the case upon stimulation of chemokine receptors that signal via a decrease in cAMP, giving rise to an increase in eYFP and thus BRET ratio.

Tailless CCL21 resembles CCL19 signalling and CCL19 chimera resembles CCL21 signalling (FIG. 4A-D). It was found that SEQ ID NO: 1 had no effect on CCL19 and tailless CCL21 induced signalling (grey curves (no TP) resembles black curves (+TP)), but potentiated the effects of chemokines CCL21 and CCL19 chimera, both containing CCL21 Tail peptide (FIG. 4A-D, black curves (+TP) show increased signalling compared to grey curves (no TP)).

### Example 3, Effect of SEQ ID NO: 1 on In Vivo Cancer Growth in Mice

SEQ ID NO: 1 was tested for its effect on in vivo cancer growth in mice. Female Balb/c mice were injected intraperitoneally with 1.5×10⁵ CT26-FL3-luc cancer cells on day 0. The CT26-FL3-luc cell line is derived from a highly metastatic murine colorectal cancer and has been engineered to express luciferase. On days 1, 2, and 3, the mice were injected intraperitoneally with either 0.25 μg murine CCL21, 20 μg human CCL21-Tail peptide (SEQ ID NO: 1), or vehicle (PBS). The CCL21 concentration was determined based on previously published in vivo experiments using CCL21. Administration of SEQ ID NO: 1 functions by a different mechanism than administration of CCL21 as SEQ ID NO: 1 affects the distribution of endogenous CCL21 in the surrounding tissue. Cancer growth was followed over time in live animals by injecting luciferin intraperitoneally and measuring the bioluminescent signal arising from the luciferase-expressing cancer cells using an In Vivo Imaging System (IVIS®) scanner. The bioluminescent signal (total flux) was used to quantify the cancer growth. The number of mice per group was 5-7, and data is shown as mean±SEM. Both CCL21 and SEQ ID NO: 1 treatment decreased tumor growth (FIG. 5). Treatment with SEQ ID NO: 1 was shown to be more effective than treatment with the chemokine CCL21.

1. A polypeptide for use in the treatment of cancer or for potentiating immune activation in a tumor or for potentiating the chemotactic potential of CCL21 having a length of less than 100 amino acids comprising or consisting of an amino acid sequence selected from the group consisting of
a) the amino acid sequence according to SEQ ID NO: 1,
b) a variant of SEQ ID NO: 1, wherein said variant has at least 80%, such as at least 90%, such as at least 95% sequence identity to SEQ ID NO: 1, but less than 99% sequence identity to SEQ ID NO: 1;
c) a variant of SEQ ID NO: 1, wherein said variant has between 1 and 10 amino acid substitutions relative to SEQ ID NO: 1, such as 1, 2, 3, 4 or 5 amino acid substitutions relative to SEQ ID NO: 1;
d) a fragment of SEQ ID NO: 1 having a length of at least 10 amino acids, or a variant thereof having between 1 and 5 amino acid substitutions relative to SEQ ID NO: 1, such as 1, 2 or 3 amino acid substitutions relative to SEQ ID NO: 1;
e) an amino acid sequence differing from SEQ ID NO: 1 by truncation at the N-terminus by at least one amino acid, such as between 1-20 amino acids, such as between 1-15 amino acids, for example between 1-10 amino acids, such as between 1-5 amino acids, or a variant thereof having between 1 and 5 amino acid substitutions relative to SEQ ID NO: 1, such as 1, 2 or 3 amino acid substitutions relative to SEQ ID NO: 1;
f) an amino acid sequence differing from SEQ ID NO: 1 by truncation at the C-terminus by at least one amino acid, such as between 1-19 amino acids, such as between 1-15 amino acids, for example between 1-10 amino acids, such as between 1-5 amino acids, or a variant thereof having between 1 and 5 amino acid substitutions relative to SEQ ID NO: 1, such as 1, 2 or 3 amino acid substitutions relative to SEQ ID NO: 1; and
g) an amino acid sequence differing from SEQ ID NO: 1 by truncation at the N-terminus by at least one amino acid, such as between 1-20 amino acids, such as between 1-15 amino acids, for example between 1-10 amino acids, such as between 1-5 amino acids, and at the C-terminus by at least one amino acid, such as between 1-19 amino acids, such as between 1-15 amino acids, for example between 1-10 amino acids, such as between 1-5 amino acids, wherein said polypeptide has a length of at least 10 amino acids, or a variant thereof having between 1 and 5 amino acid substitutions relative to SEQ ID NO: 1, such as 1, 2 or 3 amino acid substitutions relative to SEQ ID NO: 1;
wherein said polypeptide is capable of binding to glycosaminoglycans (GAGs).

2. The polypeptide for use according to claim 1, wherein said polypeptide has a length of less than 90, 85, 80, 75, 70, 65, 60, 55, 50, 45, 40, 35, 30, 25, 20 or 15 amino acids.

3. The polypeptide for use according to any of the preceding claims, wherein the polypeptide has a length of at least 10, 11, 12, 13, 14, 15, 16, 17, 18, 19, 20, 21, 22, 23, 24, 25, 26, 27, 28, 29 or 30 amino acids.

4. The polypeptide for use according to any of the preceding claims, wherein the amino acid substitutions are conservative substitutions.

5. The polypeptide for use according to any of the preceding claims, wherein the amino acid substitutions increase the net charge of said polypeptide.

6. The polypeptide for use according to any of the preceding claims, wherein the amino acid substitutions are substitutions of negative or neutral amino acid residues to positive amino acid residues.

7. The polypeptide for use according to any of the preceding claims, wherein the amino acid substitutions are substitutions of negative amino acid residues to neutral or positive amino acid residues.

8. The polypeptide for use according to any of the preceding claims, wherein said polypeptide comprises one or more moieties conjugated to said polypeptide, optionally wherein the polypeptide and the one or more moieties are conjugated to each other by a linker.

9. The polypeptide for use according to claim 8, wherein the one or more moieties are selected from the group consisting of albumin, fatty acids, polyethylene glycol (PEG), acylation groups, antibodies and antibody fragments.

10. The polypeptide for use according to claim 8, wherein the one or more moieties selected from the group consisting proteins, peptides and receptor ligands.

11. The polypeptide for use according to any of the preceding claims, wherein said polypeptide is administered in combination with an anti-cancer agent, such as for example cyclophosphamide, Paclitaxel, 5-fluorouracil, CTLA-4 antagonists, PD-L1 antagonists or PD-1 antagonists.

12. The polypeptide for use according to any of the preceding claims, wherein said polypeptide is administered in combination with radiation therapy.

13. The polypeptide for use according to any of the preceding claims, wherein said polypeptide is administered in combination with cell-based anticancer immunotherapy.

14. The polypeptide for use according to any of the preceding claims, wherein said polypeptide is administered in combination with a cytokine, such as for example CCL21, INF-γ, IL-2, CXCL9, CXCL10 or CXCL12.

15. The polypeptide for use according to any of the preceding claims, wherein said polypeptide is administered in combination with CCL21.

16. The polypeptide for use according to any of the preceding claims, wherein the cancer is a CCR7-positive cancer.

17. An isolated polypeptide having a length of less than 100 amino acids comprising or consisting of an amino acid sequence selected from the group consisting of
a) the amino acid sequence according to SEQ ID NO: 1,
b) a variant of SEQ ID NO: 1, wherein said variant has at least 80%, such as at least 90%, such as at least 95% sequence identity to SEQ ID NO: 1, but less than 99% sequence identity to SEQ ID NO: 1;
c) a variant of SEQ ID NO: 1, wherein said variant has between 1 and 10 amino acid substitutions relative to SEQ ID NO: 1, such as 1, 2, 3, 4 or 5 amino acid substitutions relative to SEQ ID NO: 1;
d) a fragment of SEQ ID NO: 1 having a length of at least 10 amino acids, or a variant thereof having between 1 and 5 amino acid substitutions relative to SEQ ID NO: 1, such as 1, 2 or 3 amino acid substitutions relative to SEQ ID NO: 1;
e) an amino acid sequence differing from SEQ ID NO: 1 by truncation at the N-terminus by at least one amino acid, such as between 1-20 amino acids, such as between 1-15 amino acids, for example between 1-10 amino acids, such as between 1-5 amino acids, or a variant thereof having between 1 and 5 amino acid substitutions relative to SEQ ID NO: 1, such as 1, 2 or 3 amino acid substitutions relative to SEQ ID NO: 1;
f) an amino acid sequence differing from SEQ ID NO: 1 by truncation at the C-terminus by at least one amino acid, such as between 1-19 amino acids, such as between 1-15 amino acids, for example between 1-10 amino acids, such as between 1-5 amino acids, or a variant thereof having between 1 and 5 amino acid substitutions relative to SEQ ID NO: 1, such as 1, 2 or 3 amino acid substitutions relative to SEQ ID NO: 1; and
g) an amino acid sequence differing from SEQ ID NO: 1 by truncation at the N-terminus by at least one amino acid, such as between 1-20 amino acids, such as between 1-15 amino acids, for example between 1-10 amino acids, such as between 1-5 amino acids, and at the C-terminus by at least one amino acid, such as between 1-19 amino acids, such as between 1-15 amino acids, for example between 1-10 amino acids, such as between 1-5 amino acids, wherein said polypeptide has a length of at least 10 amino acids, or a variant thereof having between 1 and 5 amino acid substitutions relative to SEQ ID NO: 1, such as 1, 2 or 3 amino acid substitutions relative to SEQ ID NO: 1;
wherein said polypeptide is capable of binding to glycosaminoglycans (GAGs).

18. The polypeptide according to claim 17, wherein said polypeptide consists of an amino acid sequence according to any one of SEQ ID NOs: 1-33, or a variant thereof having at least 80% sequence identity, such as at least 90%, such as at least 95% sequence identity to said sequence and having at least one amino acid substitution relative thereto, such as 1, 2 or 3 amino acid substitutions.

19. The polypeptide according to claim 17, wherein said polypeptide consists of an amino acid sequence according to any of SEQ ID NOs: 2, 3, or 5-33 or a variant of any of said sequences, wherein said variant has at least 80% sequence identity to said sequence, such as at least 90%, such as at least 95% sequence identity to said sequence, and wherein said sequence comprises at least one amino acid substitution relative to said sequence, such as 1, 2 or 3 amino acid substitutions.

20. The polypeptide according to any of claims 17-19, wherein said polypeptide has a length of less than 90, 85, 80, 75, 70, 65, 60, 55, 50, 45, 40, 35, 30, 25, 20 or 15 amino acids.

21. The polypeptide according to any of claims 17 to 20, wherein the polypeptide has a length of at least 10, 11, 12, 13, 14, 15, 16, 17, 18, 19, 20, 21, 22, 23, 24, 25, 26, 27, 28, 29 or 30 amino acids.

22. The polypeptide according to any of claims 17 to 21, wherein the amino acid substitutions are conservative substitutions.

23. The polypeptide according to any of claims 17 to 22, wherein the amino acid substitutions increase the net charge of said polypeptide.

24. The polypeptide according to claim 23, wherein the amino acid substitutions are substitutions of negative or neutral amino acid residues to positive amino acid residues.

25. The polypeptide according to claim 23, wherein the amino acid substitutions are substitutions of negative amino acid residues to neutral or positive amino acid residues.

26. The polypeptide according to any of claims 17 to 25, wherein said polypeptide comprises one or more moieties conjugated to said polypeptide, optionally wherein the polypeptide and the one or more moieties are conjugated to each other by a linker.

27. The polypeptide according to claim 26, wherein the one or more moieties are selected from the group consisting of albumin, fatty acids, polyethylene glycol (PEG), acylation groups, antibodies and antibody fragments.

28. An isolated polynucleotide encoding the polypeptide according to any of claims 17 to 27.

29. A vector comprising the polynucleotide according to claim 28.

30. The vector according to claim 29, wherein the vector is a viral vector, selected from the group consisting of adenoviruses, lentiviruses, adeno-associated viruses, herpesviruses, vaccinia viruses, poxviruses and oncolytic viruses.

31. The vector according to claim 29, wherein the vector is an expression vector, such as an expression vector selected from the group consisting of E. coli expression vector and SF9-insect expression vectors.

32. A host cell comprising the polynucleotide according to claim 28 and/or the vector according to any of claims 29 to 31.

33. A pharmaceutical composition comprising the polypeptide according to any of claims 17 to 27, the isolated polynucleotide according to claim 28, the vector according to any of claims 29 to 31, or the host cell according to claim 32.

34. The pharmaceutical composition according to claim 33, wherein the polypeptide according to any of claims 17 to 27, the isolated polynucleotide according to claim 28, the vector according to any of claims 29 to 31, or the host cell according to claim 32 is formulated in a nanoparticle.

35. The polypeptide according to any of claims 17 to 27, the isolated polynucleotide according to claim 28, the vector according to any of claims 29 to 31, or the host cell according to claim 32 for use as a medicament.

36. The polypeptide according to any of claims 17 to 27, the isolated polynucleotide according to claim 28, the vector according to any of claims 29 to 31, or the host cell according to claim 32 for use in the manufacture of a medicament for treatment of cancer.


SEQUENCE LISTING (36 sequences)

SEQ ID NO: 1 - protein, 41 aa, Homo sapiens
PSPQKPAQGCRKDRGASKTGKKGKGSKGCKRTERSQTPKGP

SEQ ID NO: 2 - protein, 31 aa, Homo sapiens
PSPQKPAQGCRKDRGASKTGKKGKGSKGCKR

SEQ ID NO: 3 - protein, 21 aa, Homo sapiens
RKDRGASKTGKKGKGSKGCKR

SEQ ID NO: 4 - protein, 31 aa, Homo sapiens
RKDRGASKTGKKGKGSKGCKRTERSQTPKGP

SEQ ID NO: 5 - protein, 31 aa, Homo sapiens
RKARGASKTGKKGKGSKGCKRTARSQTPKGP

SEQ ID NO: 6 - protein, 30 aa, Homo sapiens
KDRGASKTGKKGKGSKGCKRTERSQTPKGP

SEQ ID NO: 7 - protein, 29 aa, Homo sapiens
DRGASKTGKKGKGSKGCKRTERSQTPKGP

SEQ ID NO: 8 - protein, 28 aa, Homo sapiens
RGASKTGKKGKGSKGCKRTERSQTPKGP

SEQ ID NO: 9 - protein, 27 aa, Homo sapiens
GASKTGKKGKGSKGCKRTERSQTPKGP

SEQ ID NO: 10 - protein, 26 aa, Homo sapiens
ASKTGKKGKGSKGCKRTERSQTPKGP

SEQ ID NO: 11 - protein, 25 aa, Homo sapiens
SKTGKKGKGSKGCKRTERSQTPKGP

SEQ ID NO: 12 - protein, 24 aa, Homo sapiens
KTGKKGKGSKGCKRTERSQTPKGP

SEQ ID NO: 13 - protein, 23 aa, Homo sapiens
TGKKGKGSKGCKRTERSQTPKGP

SEQ ID NO: 14 - protein, 22 aa, Homo sapiens
GKKGKGSKGCKRTERSQTPKGP

SEQ ID NO: 15 - protein, 21 aa, Homo sapiens
KKGKGSKGCKRTERSQTPKGP

SEQ ID NO: 16 - protein, 20 aa, Homo sapiens
KGKGSKGCKRTERSQTPKGP

SEQ ID NO: 17 - protein, 30 aa, Homo sapiens
RKDRGASKTGKKGKGSKGCKRTERSQTPKG

SEQ ID NO: 18 - protein, 29 aa, Homo sapiens
RKDRGASKTGKKGKGSKGCKRTERSQTPK

SEQ ID NO: 19 - protein, 28 aa, Homo sapiens
RKDRGASKTGKKGKGSKGCKRTERSQTP

SEQ ID NO: 20 - protein, 27 aa, Homo sapiens
RKDRGASKTGKKGKGSKGCKRTERSQT

SEQ ID NO: 21 - protein, 26 aa, Homo sapiens
RKDRGASKTGKKGKGSKGCKRTERSQ

SEQ ID NO: 22 - protein, 25 aa, Homo sapiens
RKDRGASKTGKKGKGSKGCKRTERS

SEQ ID NO: 23 - protein, 24 aa, Homo sapiens
RKDRGASKTGKKGKGSKGCKRTER

SEQ ID NO: 24 - protein, 23 aa, Homo sapiens
RKDRGASKTGKKGKGSKGCKRTE

SEQ ID NO: 25 - protein, 22 aa, Homo sapiens
RKDRGASKTGKKGKGSKGCKRT

SEQ ID NO: 26 - protein, 21 aa, Homo sapiens
RKDRGASKTGKKGKGSKGCKR

SEQ ID NO: 27 - protein, 20 aa, Homo sapiens
RKDRGASKTGKKGKGSKGCK

SEQ ID NO: 28 - protein, 19 aa, Homo sapiens
RKDRGASKTGKKGKGSKGC

SEQ ID NO: 29 - protein, 18 aa, Homo sapiens
RKDRGASKTGKKGKGSKG

SEQ ID NO: 30 - protein, 17 aa, Homo sapiens
RKDRGASKTGKKGKGSK

SEQ ID NO: 31 - protein, 16 aa, Homo sapiens
RKDRGASKTGKKGKGS

SEQ ID NO: 32 - protein, 15 aa, Homo sapiens
RKDRGASKTGKKGKG

SEQ ID NO: 33 - protein, 14 aa, Homo sapiens
RKDRGASKTGKKGK

SEQ ID NO: 34 - protein, 134 aa, Homo sapiens
MAQSLALSLLILVLAFGIPRTQGSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 35 - protein, 111 aa, Homo sapiens
SDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 36 - protein, 21 aa, Homo sapiens
PSPQKPAQGCRKDRGASKTGK